(19) United States
(12) Patent Application Publication
(10) Pub. No.: US 2021/0401495 A1
(43) Pub. Date: Dec. 30, 2021

(54) METHOD AND SYSTEM FOR MONITORING TISSUE ABLATION THROUGH CONSTRAINED IMPEDANCE MEASUREMENTS

(71) Applicants: The University of Sydney, New South Wales (AU); Western Sydney Local Health District, New South Wales (AU)

(72) Inventors: Pierre Qian, New South Wales (AU); Michael Anthony Barry, New South Wales (AU); Alistair McEwan, New South Wales (AU); Duc Minh Nguyen, Hanoi (VN)

(73) Assignees: The University of Sydney, New South Wales (AU); Western Sydney Local Health District, New South Wales (AU)

(21) Appl. No.: 17/468,420

(22) Filed: Sep. 7, 2021

Related U.S. Application Data
(63) Continuation of application No. PCT/AU2020/050325, filed on Apr. 2, 2020.

(30) Foreign Application Priority Data
Apr. 2, 2019 (AU) 2019901118

(51) Int. Cl. A61B 18/14; A61B 18/12

(52) U.S. Cl. CPC A61B 18/1492; A61B 2018/00577; A61B 18/1206

(57) ABSTRACT

A system for monitoring tissue lesion development during a medical ablation process applied to a patient, the system comprising a catheter ablation device having at least one catheter electrode, the device connectable via an electrical feedline to a source of electrical energy and configured to apply ablation energy to ablate tissue in a target region, a plurality of external electrodes for application to the body of the patient, measurement circuitry for determining an electrical characteristic of a current path between the at least one catheter electrode and the external electrodes in the absence of said application of ablation energy, and an electrical controller. The system can be used for monitoring the size of a lesion during a catheter ablation process applied to the tissue of a subject, comprising alternating between an ablation phase involving delivery of ablation energy to a catheter electrode and a measure phase involving measuring an electrical characteristic of a current path passing through a lesion area formed by the ablation, in which the two phases are sequentially repeated until analysis of the measurement results indicate attainment of a desired lesion size.

### CROSS-REFERENCES TO RELATED APPLICATIONS

This application claims priority to and is a continuation of International Patent Application No. PCT/AU2020/050325, filed Apr. 2, 2020; which claims priority from AU Patent Application No. 2019901118, filed Apr. 2, 2019. The entire contents of each of the PCT/AU2020/050325 and the AU Patent Application No. 2019901118 are hereby incorporated by reference in their entirety for all purposes.

### FIELD OF THE INVENTION

The invention relates to a method and system for monitoring tissue ablation through constrained impedance measurements. It has particular application in real-time continuous evaluation of intravascular cardiac catheter ablation treatments, but may equally find application in a variety of other medical treatment techniques.

### BACKGROUND OF THE INVENTION

Cardiac catheter ablation such as radiofrequency (RF) ablation is capable of treating a wide range of cardiac arrhythmias in a minimally invasive way, and constitutes a rapidly growing field in interventional cardiology. Regions of the heart involved with these arrhythmias can be reached via access from a peripheral vein or artery with a catheter equipped with a suitable ablation device (such as an RF radiation electrode or other suitable instrument) and can be ablated by applying the ablation energy to heat the tissue.

RF catheter ablation involves the delivery of high frequency alternating electrical current (in the range 350 kHz to 1 MHz) through one or more electrode catheters to myocardial tissue to create a thermal lesion. The mechanism by which the current heats the tissue is resistive (ohmic) heating of a narrow rim (<1 mm) of tissue in direct contact with the electrode, with deeper tissue regions heated by conduction. Heat is dissipated from the region by further heat conduction into normothermic tissue and by heat convection via the circulating blood pool.

A lesion that is too small may be ineffective in treating the arrhythmia, while lesions that are too large can be associated with unwelcome complications. Overheating in this area is a major concern, with potential risks including puncture and tamponade. Successful catheter ablation thus requires not only precise localization of the arrhythmogenic substrate but complete and permanent elimination of that substrate, without producing collateral injury.

Despite the need for monitoring lesion development during ablation procedures, there are presently no reliable means for achieving this clinically. Surrogate measures such as catheter tip temperature and impedance changes, ablation power, duration, catheter tip pressure and diminution of intracardiac electrograms recorded on the ablation catheter can provide an indication that the catheter tip is appropriately located relative to the cardiac wall and that ablation is occurring, but are generally not able to offer any direct measure of lesion formation or development. MM can provide high resolution images of lesions with relatively small errors, but image reconstruction time is high (up to 30 minutes) and the technique therefore not practicable for standard clinical procedures.

Previous systems for determining lesion size include the use of Electrical Impedance Tomography (EIT). EIT suffers in its traditional implementation, as it is an ill-posed method that produces low spatial resolution results. EIT implemented systems therefore rely heavily on CT imaging or position information throughout treatment, which is utilized in addition to real-time catheter position knowledge.

A more reliable system and method for monitoring lesion development during catheter ablation is required, without the numerical solutions of EIT or the need for recourse to CT information.

Reference to any prior art in the specification is not an acknowledgment or suggestion that this prior art forms part of the common general knowledge in any jurisdiction or that this prior art could reasonably be expected to be combined by a skilled reader with any other pieces of prior art.

### BRIEF SUMMARY OF THE INVENTION

During delivery of electromagnetic radiation in a catheter ablation procedure, temperature changes in cardiac tissue due to resistive and conduction heating are accompanied by a change in the electrical impedance of the tissue. Theoretically, impedance drops when temperature increases. This permits a real time measurement of heating in a volume of tissue by measuring the changed impedance of the tissue volume.

In accordance with the invention in a first aspect, there is provided a system for monitoring tissue lesion development during a medical ablation process applied to a patient, the system comprising: a catheter ablation device having at least one catheter electrode; the device connectable via an electrical feedline to a source of electrical energy and configured to apply ablation energy to ablate tissue in a target region; a plurality of external electrodes for application to the body of the patient; measurement circuitry for determining an electrical characteristic of a current path between the at least one catheter electrode and the external electrodes in the absence of said application of ablation energy; and an electrical controller.

Preferably, the electrical characteristic is the impedance of the current path.

In a preferred form, the electrical controller is arranged to control application of an AC current source between different combinations of the at least one catheter electrode and the plurality of external electrodes such that measurement of the resulting voltages provides a measure of impedance of different electrical paths through the body of the patient between the respective electrodes and the electrical controller is further configured to disconnect the catheter ablation device from the source of electrical energy or otherwise suspend said application of ablation energy during application of said AC current source.

The system may include a dummy resistive load for selective connection to the source of electrical energy during periods of operation of said measurement circuitry. In this case, an ablation shunt may be included, configured to uncouple the source of energy source from the catheter ablation device and couple it to the dummy load.

Alternatively, an intermittent source of electrical energy may be used, which can be rapidly switched off for the periods when the measurement are being made.

In a preferred form, the measurement circuitry includes a switch matrix arranged for switching between the different combinations of electrodes under control of the electrical controller.

Preferably, the measurement circuitry is configured to conduct four-terminal sensing to measure said electrical characteristic (e.g., the impedance).

The electrical controller may comprise a PC. In a preferred form, the measurement circuitry includes one or more analog-to-digital converters (ADC) to provide a digital representation of measured voltage. In one embodiment, multiple ADCs are included, for simultaneous measurement of different current paths, each ADC arranged to be switched between different selected external electrodes under control of the electrical controller.

In one form, the source of electrical energy is an RF generator. The invention may also be applied to other types of ablation processes, including microwave ablation and electroporation.

The plurality of external electrodes may be provided as an electrode dot harness for application across an external area of the patient's body.

In accordance with the invention in a second aspect, there is provided a method of operating a system for monitoring the size of a lesion during a catheter ablation process applied to the tissue of a subject, the method comprising: (a) performing an ablation phase involving delivery of ablation energy to a catheter electrode; (b) performing a measure phase involving measuring an electrical characteristic of a current path passing through a lesion area formed by the ablation: wherein steps (a) and (b) are sequentially repeated.

In a preferred form, steps (a) and (b) are sequentially repeated until the measurements performed in step (b) indicate a prescribed lesion size.

In step (b), ablation energy may be diverted from the catheter electrode to a dummy load.

The method of the second aspect of the invention may include use of the system of the first aspect of the invention, wherein step (a) is conducted using said catheter ablation device and step (b) is conducted using said plurality of external electrodes and said measurement circuitry, the switching between steps (a) and (b) made under control of said electrical controller.

Hence, in accordance with the method, the measure phase involves passing an electrical current sequentially between one or more catheter electrodes and a plurality of electrodes applied externally of the body of a patient and measuring the electrical response. Analysis of the results affords an evaluation of the effect of the most recent ablation phase, and analysis of the results of successive measure phases allows a prediction with regard to attainment of desired lesion size.

The method may include an initial determination phase in which one or more current paths are selected from a plurality of current paths by applying an electrical current sequentially between one or more catheter electrodes and a plurality of external electrodes applied to the body of a patient and measuring the electrical response, and selecting the electrodes to use for step (b) in accordance with the results.

Preferably, a prescribed number of current paths are selected in the determination phase, with the associated electrodes used for subsequent iterations of step (b).

In one embodiment, the electrodes are selected as those associated with the lowest impedance of the current paths measured. Alternatively, the electrodes may be selected as those associated with the current paths most sensitive to a local state change of the body of the patient, such as the injection of conducting solution to a region adjacent the lesion.

The change in impedance may be compared with previously determined data (e.g., in a look-up table) to provide to the medical practitioner a measure of lesion size. Tests have suggested that the method of the invention can be used to track lesion size within an error of only around 1 mm in depth and 3 mm in length, seen to be clinically acceptable in most applications.

In one preferred form, the method includes using the measurements made in step (b) in an algorithm to estimate the size of the lesion formed in step (a).

In one embodiment, for each measure phase, the measurement results are analyzed and a selection is made as to which measurements to use in the algorithm.

This selection may be made based at least in part on the change in the electrical characteristic of the relevant current paths since the previous measure phase. For example, the selection may be made based on the largest impedance drop caused by the intervening ablation phase.

In a preferred form, step (a) and/or step (b) may be gated to the respiration cycle and/or the heartbeat of the subject, in order to carry out the measure phase at a relatively stable point.

The algorithm used in the analysis of the measurements may include a regression analysis algorithm. Alternatively or in addition, machine learning may be used to interpret the results. As will be understood, the analysis of the results (based in particular on the position of the external electrodes used for each measurement) may be used in determination of lesion dimensions, lesion shape and/or lesion orientation.

The present invention therefore involves impedance measurements between ablation catheter electrodes and a plurality of external electrodes. In this specification and claims the term ‘external electrodes’ is used to refer to a secondary set of electrodes remote from the catheter. In common applications, the external electrodes are placed externally of and in contact with the patient's body. However it will be understood that they may be placed within internal structures of the body such as the oesophagus, coronary sinus or other suitable sites. The catheter electrodes and external electrodes are used to rapidly and reliably find the most clinically significant current paths and to obtain a measure of the impedance changes as the ablation progresses, which can provide a clinically useful indication of the growth of the lesion.

The use of multiple impedance measurements between a plurality of electrodes in different locations on a patient's body is of course known in the general field of EIT. However EIT is used for medical imaging, with particular application in areas such as monitoring lung function, location of cancerous regions, localization of brain activity and gastric activity. In contrast, the present invention does not rely on image reconstruction software, but instead uses a combination of the electrode(s) comprised in the ablation catheter with a plurality of external electrodes, along with a specially-configured switching means, to determine which of the electrode groupings (corresponding to particular conduction paths) to use in ongoing monitoring of the effectiveness of the use of the ablation catheter, the response in the measured electrical characteristics of those current paths providing a relatively direct, real-time indication of the progress of lesion formation. Like EIT, the currents typically applied in the method of the invention are relatively small and at a suitably high frequency to avoid significant nerve stimulation or ohmic heating within the body. Unlike the use of EIT to monitor lesion formation, the present invention does away with the need for complex computational solutions, and also the need for recourse to CT imaging or position information.

The proximity of the catheter electrode(s) establishes inclusion of the heated volume in the resulting electrical path to one or more external electrodes and, in accordance with the invention, the most appropriate current paths are found by iterating application of current over the plurality of electrodes and performing voltage measurements. Prescribed criteria (such as the lowest calculated impedance measurements) are considered as indication of the most appropriate paths for monitoring of lesion formation during ablation treatment.

### BRIEF DESCRIPTION OF THE DRAWINGS

Further aspects of the present invention and further embodiments of the aspects described in the preceding paragraphs will become apparent from the following description, given by way of example and with reference to the accompanying drawings, in which:

FIG. 1 is an overview of a system for monitoring lesion development during RF catheter ablation of a patient, according to one embodiment of the present invention;

FIG. 2 depicts an ablation interface of the system of FIG. 1 connected to an RF generator;

FIG. 3 depicts an alternative interface the system;

FIG. 4 is a flow diagram illustrating a method for monitoring lesion development during catheter ablation, in accordance with one embodiment of the present invention;

FIG. 5 is a flow diagram of the measurement phase of the method illustrated in FIG. 4;

FIG. 6 is a flow diagram illustrating a method for monitoring lesion development during catheter ablation in accordance with an alternative embodiment of the present invention;

FIG. 7 is a flow diagram of the measurement phase of the method illustrated in FIG. 6;

FIG. 8 shows an embodiment of 64 ECG electrodes (dot electrodes'), arranged in four bands of 16; and

FIG. 9 is a schematic illustration of a catheter device and ablation lesion.

### DETAILED DESCRIPTION OF THE INVENTION

It will be understood that the invention disclosed and defined in this specification extends to all alternative combinations of two or more of the individual features mentioned or evident from the text or drawings. All of these different combinations constitute various alternative aspects of the invention.

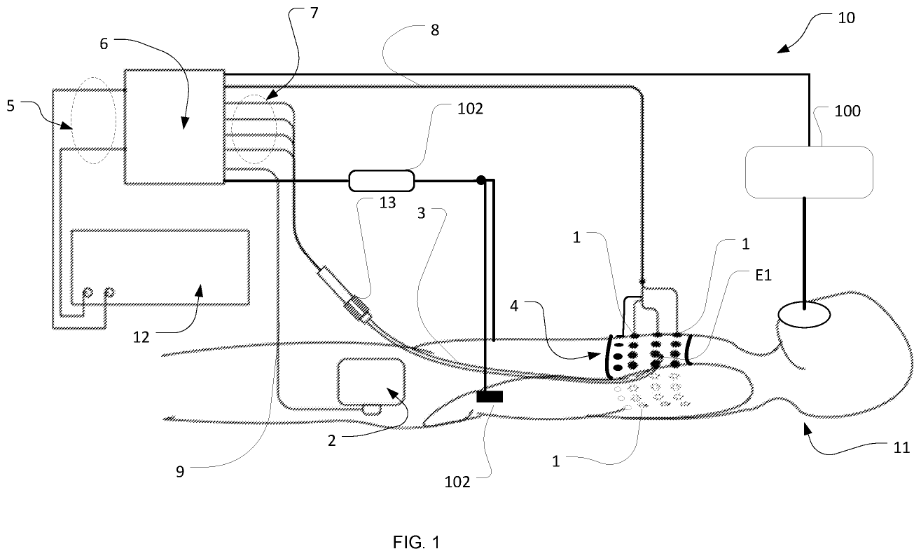

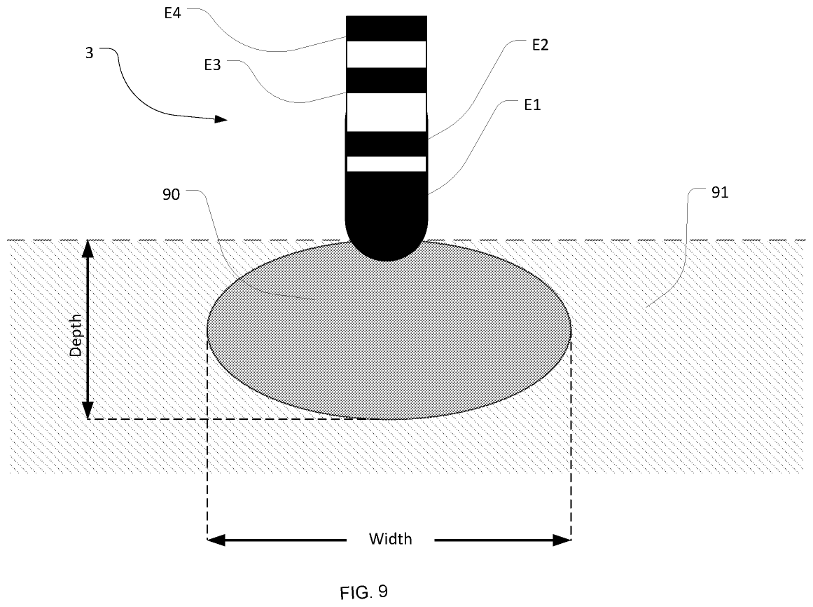

The system 10 illustrated in FIG. 1 affords monitoring of lesion development during RF catheter ablation and includes an RF ablation catheter 3 (including an RF radiator and RF power supply line) for introduction into a heart chamber of a patient 11. Catheter 3 is provided with catheter electrodes E1, E2, E3, E4, electrode E1 comprising the RF ablation electrode (see FIG. 9), while a patient return electrode 2 is attached to the patient's thigh or other suitable location. As discussed further below, a band 4 of external surface electrodes 1 is wrapped around the patient's chest. The external electrodes 1 may be conventional ECG dot electrodes, used in this case to measure voltage.

The ablation catheter 3 may be, for example, a 3.5 mm Fr Thermocool catheter (Biosense Webster Inc.), a Therapy Cool Flex ablation catheter, or any other suitable device known to those skilled in the art. The ablation generator 12 may be, for example, a Stockert 70 cardiac ablation radiofrequency generator St4520 (Biosense Webster Inc.).

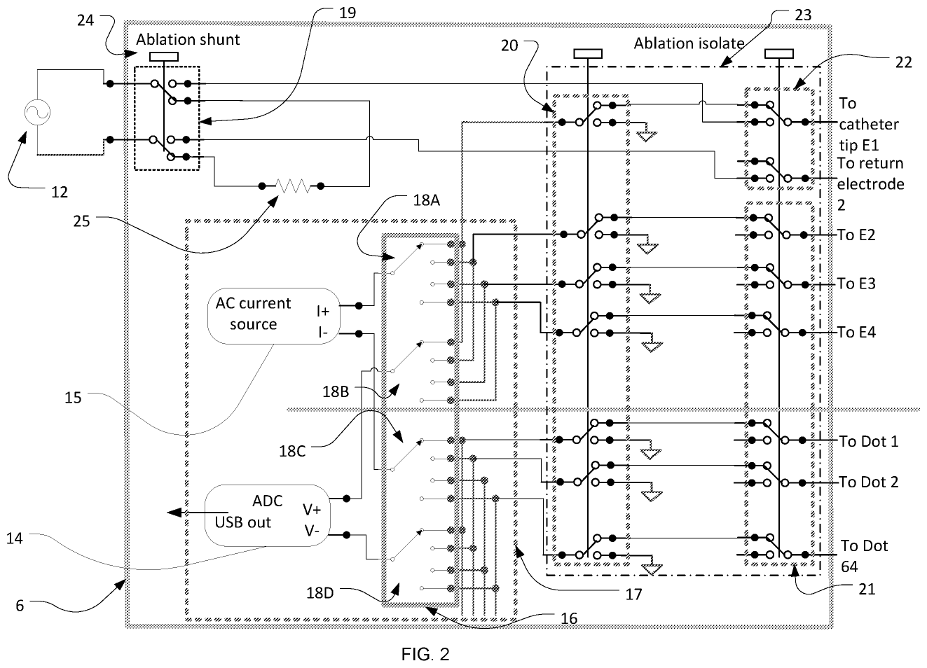

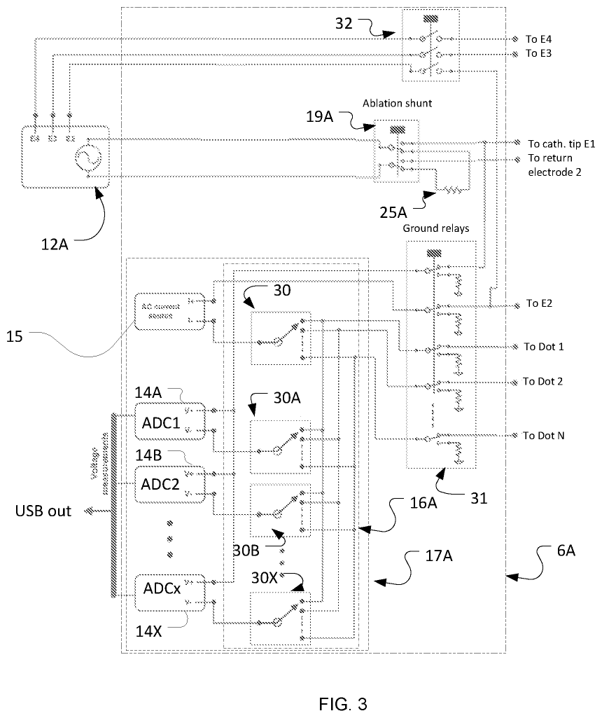

An electrical interface module 6 (also referenced as 6A with respect to Embodiment 2 of the invention, discussed further below) includes a plurality of relays and N-way switches (for example switch matrix 16/16A, comprised in impedance measuring circuit 17/17A—see FIGS. 2 and 3) configured to govern ablation and measurement phases of the treatment of patient 11.

Switching control is provided by a PC running a custom computer program (not shown). The output of RF generator 12 is referenced as input 5 to interface module 6/6A. Further, interface module 6/6A is electrically connected by a lead wire 9 to patient return electrode 2, by external electrode lead wires 8 to each external electrode 1 of electrode band 4, and by lead wires 7 to each internal electrode E1, E2, E3, E4 of catheter 3, by way of cable connector 13.

Additionally, the system may also include a real-time ECG/QRS (heartbeat) detector 102 with an ECG electrode 101 placed on each of the patient's wrists. A ventilator 100 may be used to ventilate the anaesthetized patient 11 during the ablation procedure, in which case ventilator 100 is configured such that breath cycle measurements are received by the computer program. Alternatively, if the patient 11 is under sedation only, a signal indicating respiratory function received from another source may be used, for example fluctuations in chest wall impedance.

### Embodiment 1

A first embodiment of the circuitry of electrical interface module 6 is shown in FIG. 2, connected to RF generator 12. An ablation shunt 24, relays 19 and the relays of relay groups 20, 21, 22 (collectively, relay group 23) are shown in an impedance measurement position. The N-way switches of switch matrix 16 are shown set at an arbitrary position, however during a ‘measurement phase’, the switches will cycle through multiple positions as described in detail below.

Switch matrix 16 consists of four N-way steering switches 18A,18B,18C,18D. In an example configuration, switches 18A and 18B are 4-way switches, with the throws of each switch affording connection to each of the catheter electrodes E1-E4. Switches 18C and 18D are 64-way switches, but for ease of depiction, only four terminals are shown. The throws of switches 18C and 18D afford connection to each of the 64 external surface electrodes 1. Together, these N-way steering switches 18A,18B,18C,18D allow an AC constant current source 15 and the terminals of a high-precision voltmeter 14 (with an output via an ADC) to be selectively connected across any one of the catheter electrodes E1-E4 and external electrodes 1.

An appropriate frequency of operation of the AC current source 15 is used, as determined on the basis of competing factors. The frequency must be sufficiently high to avoid tissue stimulation and to allow acquisition of several cycles of measurement in a short time period, but sufficiently low so as to minimize the effect of parasitic capacitance within the catheter and to minimize any interference from the frequency of application of ablation energy. In initial tests the inventors found that a frequency in the range 50 kHz-100 kHz was preferred. The amplitude of current injected is also selected as appropriate, as determined by competing factors. A higher current provides for better voltage resolution, especially for low impedance paths, however the current should not be so high that the electrodes themselves begin to heat. In initial tests the inventors found that a current in the range 2-5 mA was preferred.

Measuring circuit 17 is thus configured to perform sequential four-terminal impedance measurements. To perform each measurement, current is supplied between a first catheter electrode E1/E2/E3/E4 and a first external electrode 1, and the resulting voltage is measured between a second of the catheter electrodes and a second external electrode, neighboring the first external electrode. The resulting impedance is then passed to an external PC (not shown) from the USB output of ADC voltmeter 14.

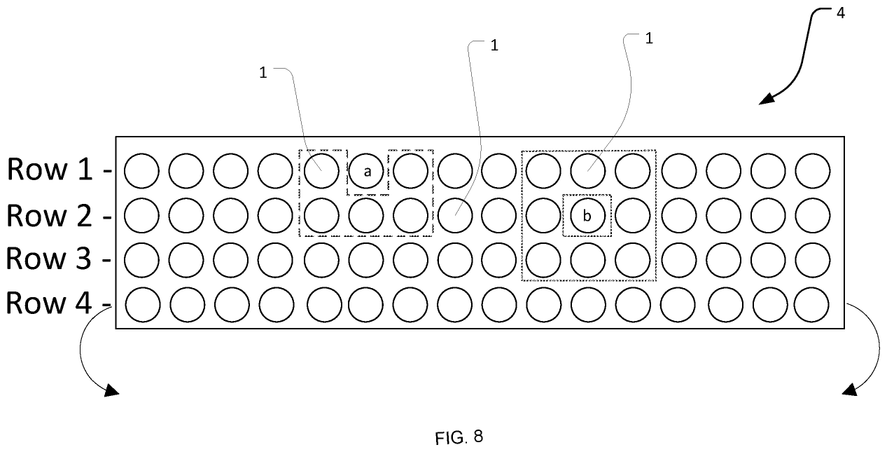

In the example configuration shown in FIG. 8, an electrode band 4 consists of four rows of 16 external ‘dot’ electrodes 1. The set of electrodes directly adjacent to electrodes ‘a’ and ‘b’ are indicated by the dashed and dotted outlines respectively. As will be noted, electrode ‘a’ (as all other electrodes in the upper or lower rows) has five direct neighbor electrodes, while electrode ‘b’ (as all other electrodes in the central rows) has eight. Electrode band 4 is shown flat in FIG. 8, but it will be understood that in use it is wrapped around the patient's chest, such that the depicted left-most and right-most electrodes become mutually neighboring electrodes.

As an example four-terminal arrangement, obtaining an impedance measurement in a conduction path between catheter 3 and electrode ‘a’ is achieved by connecting the positive terminal I+ of current source 15 to catheter electrode E3, the negative terminal I− of current source 15 to external electrode ‘a’, the positive terminal V+ of ADC voltmeter 14 to catheter electrode E2, and the negative terminal V− of ADC voltmeter 14 to any one of the five external electrodes 1 neighboring electrode ‘a’. Hence any of five measurements may provide a determination of a current path to the catheter associated with electrode ‘a’, and the method of the present invention uses all five measurements to determine the most suitable. The same applies for any electrode in the upper or lower rows of electrode band 4.

Similarly, for electrode ‘b’ (or any other electrode in either of the middle rows of electrode band 4), any of eight measurements may provide a determination of a current path to the catheter associated with that electrode, and the method of the present invention uses all eight measurements to determine the most suitable. Impedance measurements are discussed further below with reference to the calibration and measurement phases of the method of the invention.

Returning to FIG. 2, ablation shunt 24 consists of two SPDT (single-pole double-throw) relays 19, which operate simultaneously to either direct electrical ablation power from RF generator 12 across catheter electrode E1 and return electrode 2, or across a dummy load 25 (for example a 10 Ω resistor) while measurements are being performed. The SPDT relays 19 may be for example G6EK-134P-ST-US-DC5 (Omron Electronics Components) relays. This arrangement provides protection of measuring circuit 17 and other componentry from high voltage and from RF noise.

Further, ablation isolate relay groups 20, 21, 22 (collectively referenced as relay group 23) are arranged to operate synchronously with ablation shunt relays 19. During ablation, grounding relays 20 connect the throws of the N-way switches of switch matrix 17 to ground. Isolate relays 21 isolate external dot electrodes 1 and catheter electrodes E2 to E4. Relays 22 connect catheter tip electrode E1 and return electrode to respective throws of the ablation shunt relays.

In one state (in which impedance measurements can be made), relay groups 20 and 21 together allow connections from the throws of switches 18A,18B to each of catheter electrodes E1-E4 and connections from the throws of switches 18C,18D to each of the external electrodes 1, while the return electrode 2 is disconnected from the catheter tip electrode E1 (as illustrated).

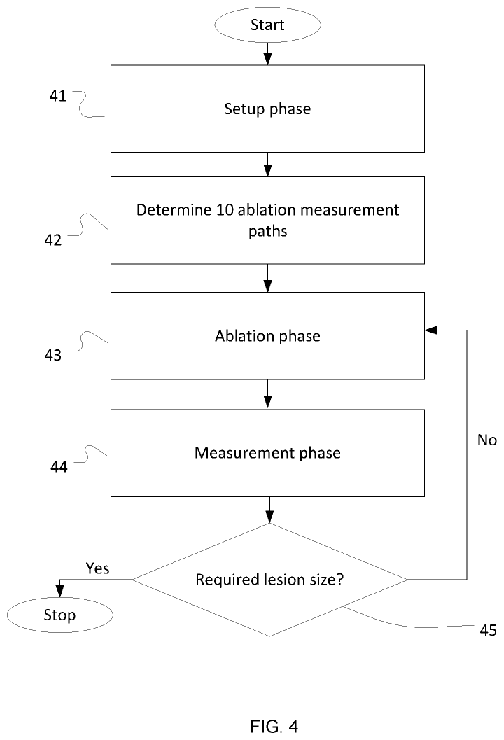

The ablation shunt relays 19 and ablation isolate relays of relay group 23 therefore enable the system to switch between two states, namely an ablation state and a measuring state. The method of the invention involves an iterative process of cycling between these two states, the present embodiment of which is discussed below with reference to FIG. 4.

The process illustrated in FIG. 4 involves a setup phase, followed by a determination phase, followed by repeated ablation and measurement phases which continue until the required lesion size is achieved (as determined using voltage/impedance measurements), at which point the treatment is stopped.

### Setup Phase

The first step of the process is the setup phase 41, during which AC current source 15 and ADC voltmeter 14 are used to obtain four-terminal internal-to-external voltage measurements using two electrodes of catheter 3 and each one of the external electrodes 1. The purpose of the setup phase is to acquire measurements for all of the possible electrical paths between the internal and external electrodes, to allow determination of the optimum paths for ongoing measurement. As will be understood, for the injection of a known current, the measured voltage provides a determination of the impedance of the current path.

As discussed above, voltage measurements resulting from the applied current are obtained for electrical paths between the catheter and external electrodes. Ablation catheters (for example the Biosence Webster Thermocool ablation catheter) commonly have four catheter electrodes, however only two internal electrodes are required for the four-terminal voltage measurements. In the example described, E2 and E3 are used for performing measurements, with E1 only used for ablation and E4 not used. E4 was considered by the inventors as too remote from the catheter tip, while tests showed that in practice impedance measurement results using E1 tended to be undesirably noisy, possibly due to a limitation in the isolation provided by ablation shunt 24 from the RF signal to E1.

Returning again to FIG. 2, to obtain four-terminal impedance measurements, I+ is connected to catheter electrode E3, V+ to catheter electrode E2, I− to a first external electrode 1, and V− sequentially to each one of the electrodes adjacent that first external electrode. Resulting voltage measurements are recorded for each. I− then is switched to connect to a second external electrode 1, with V− switching sequentially to the electrodes neighboring that second external electrode. This continues until current has been applied, and resulting voltage measured and recorded, for all of the external electrodes.

As described above, multiple four-terminal voltage measurements are taken for each of the external electrodes 1 involving the electrodes which neighbor each one. In the configuration illustrated in FIG. 8 (band 4 consisting of 64 electrodes arranged in four rows of 16), a total of 416 impedance measurements and paths are recorded (five for each electrode in the top and bottom band, and eight for each electrode in the middle bands).

### Determining Ablation Measurement Paths

Returning to FIG. 4, the process then passes to a determination step 42 in which the results from the setup phase are analyzed to make the decision as to the 10 most suitable catheter-to-external electrode paths.

Since lower impedance generally indicates a more direct path and an associated lower noise risk, the ‘best’ paths are taken to be the paths of lowest impedance. However it will be understood that in alternative approaches other criteria can be used.

For example, paths may be chosen that demonstrate highest sensitivity to the introduction of a suitable saline solution to the catheter site.

As discussed further below, rather than selecting a single internal-to-external electrode path, step 42 involves the determination of 10 paths, so that if a path is found to be unreliable (for example due to the presence of a lung field) other measurement paths are available. As the skilled reader will understand, any number of internal to external electrode paths could be selected, with the inventors determining that ten paths provides an appropriate and practicable number of alternatives for the methodology of the present invention. As will be understood, selecting more paths will involve a longer monitoring time, while selecting fewer paths may introduce stochastic errors.

In an alternative approach, discussed below with respect to Embodiment 2 of the present invention, rather than determining a limited number of paths for impedance measurement during the ablation procedure, all the path impedances may be measured in each measurement phase, with determination of which paths to use in analysis made in accordance with prescribed criteria.

Ablation phase

Once determination step 42 is complete, the RF ablation treatment commences (ablation phase 43). As discussed above, during this phase ablation isolate relays 21—under control of the PC—disconnect the catheter electrodes and external electrodes 1 from the impedance measuring circuit 17. Ablation isolate ground relays 20 connect catheter electrode and external electrode terminals of N-way switches 18 to ground.

Ablation shunt relays 19 and ablation isolate relay group 22 provide RF ablation energy from RF generator 12 to catheter tip radiator electrode E1, patient return electrode 2 providing the electrical return path. The application of the RF ablation energy for a suitable time thus heats the tissue to begin the lesion formation. In experimental tests a duration of ablation of 5.2 seconds was selected, chosen in accordance with various factors including the respiratory rate of the patient, as discussed in more detail below.

Following each ablation phase, relay circuits are used to switch RF generator 12 away from catheter tip electrode E1 and patient return electrode 2 to dummy load 25; a pause of 50 ms while this switching occurs provides time for the area surrounding the developing lesion to thermally equilibrate. During this time, the peripheral vein or artery fluids/blood heated during the ablation stage flows away from the catheter tip region, so that any thermal change only resides in the lesion.

### Measurement Phase

The measurement phase 44 is used to measure resulting voltage as current is applied from internal to external electrodes of selected paths as the ablation treatment progresses, i.e., between successive ablation cycles, so providing a measure of the size of the lesion. Following the 50 ms delay at the end of the ablation phase, RF generator 12 is switched away from catheter tip electrode E1 and patient return electrode 2 to dummy load 25.

Under control of the PC, switch matrix 16 forms connections to enable successive four-terminal voltage measurements to be made for the 10 measurement paths selected in determination phase 42.

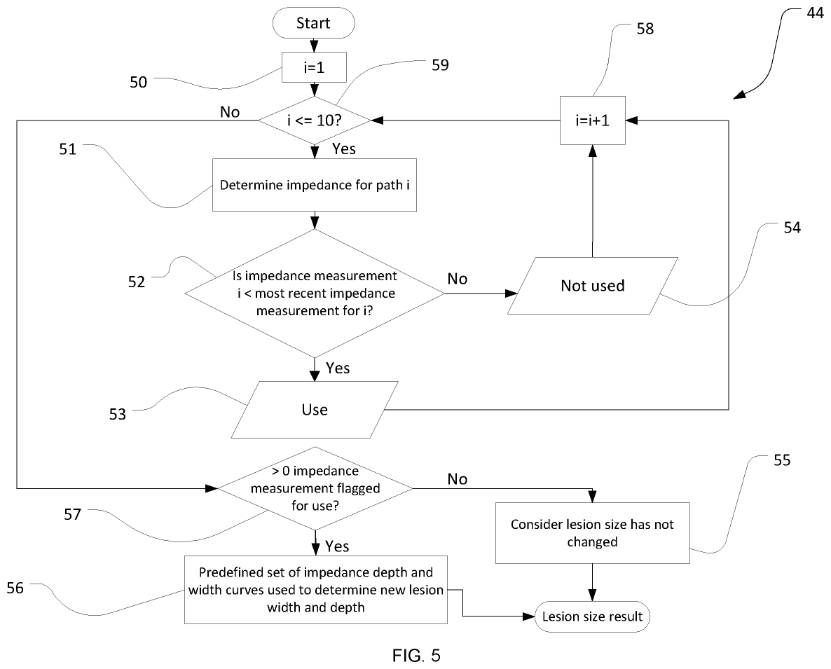

The flow diagram of FIG. 5 provides further detail of the measurement phase 44. Since the tissue impedance will drop with the increasing temperature due to ablation within the tissue, if any of the 10 impedance measurements show an increase in impedance between the present and most recent measurement (either in the setup phase or the most recent measure phase), the impedance value should be disregarded. An increase in impedance can indicate that the path has a low signal to noise ratio (SNR), or that the measurement was dominated by unexpected events or noise.

Referring to FIG. 5, the measurement phase process begins with respect to a first measurement path, i.e. path i=1 (step 50). Under control of the PC, switch matrix 16 is configured to take a single four-terminal voltage measurement for a first path identified in the decision making step 42 (step 51), which is used to determine impedance. This value is then compared with the stored previous value in a decision step 52. If this new impedance measurement for the first path is lower than the previous value, then the measurement will be used (step 53) as an indication of the size of the lesion. If the new impedance measurement is higher than the previous value, the value is discarded (step 54).

The next path is then subjected to measurement and determination of impedance, by incrementing the count (i=i+1; step 58) and repeating the process. Decision step 59 determines whether all 10 paths have been measured, in which case the process moves to decision step 57. If none of the 10 impedance values is determined to be lower than its previous measurement, the lesion may be considered the same size as the previous cycle (step 55). This may indicate that the ablation has failed and needs to be repeated, however towards the end of the ablation process, an equilibrium state is reached and the lesion no longer grows significantly. Naturally the decision as to continue ablation will be made by the cardiologist/surgeon, informed by the impedance measurement results.

If it is determined (step 57) that at least one of the impedance measurements has been flagged for use (i.e. the impedance for that particular path had decreased, indicating an increase in lesion size), then the PC uses the impedance measurements to make a determination of lesion size using a predefined set of impedance depth and width curves (step 56).

In order to exclude possible under-predicted and over-predicted values, quantiles of the sets of depths and widths for the cumulative probability of 0.45 to 0.55 are used to constrain the results. These can be extended to 0.35 to 0.65 and then 0.25 to 0.75 at maximum until at least one measurement is found falling within the range. The final measured lesion dimensions will therefore represent averaged depths and widths.

FIG. 9 provides a diagrammatic illustration of catheter 3 with electrodes E1, E2, E3, E4 in proximity with lesion 90 within tissue 91, the lesion width and height determined by the method of the invention.

Returning to FIG. 4, following measurement phase 44, a determination of the lesion size following the latest ablation cycle is made. At decision step 45, if the lesion is determined to be of the required size, the ablation treatment process ends. If the required lesion size is not yet achieved, the surgeon/cardiologist can decide to commence the next iteration of ablation, the process thus returning to ablation phase 43.

As will be understood, this repeating alternation of ablation and impedance measurement cycles affords real-time continuous monitoring of the treatment process, but without the RF field interfering with the measurement apparatus (or vice versa).

### Embodiment 2

In this alternative embodiment, the circuitry of electrical interface module 6A is shown in FIG. 3, connected to an RF generator 12A. Ablation shunt relays 19A and the relays of relay groups 31 and 32 are shown in an impedance measurement position. The N-way switches of switch matrix 16A are shown set to an arbitrary position, however during a measurement phase, the switches will again cycle through multiple positions.

Switch matrix 16A consists of a plurality of N-way steering switches 30 and 30A to 30X. Unlike the arrangement of Embodiment 1, rather than connecting to a four-way switch, I+ of the current source is connected only to electrode E2 via an ablation isolate relay, and V+ is connected only to the catheter tip electrode E1, similarly via an ablation isolate relay. The pole of steering switch 30 is connected to the I− terminal of the current source, and the poles of switches 30A to 30X are connected to the V− terminals of a plurality of analog-to-digital converters

In this embodiment, refinement of the apparatus by the inventors—in particular in providing more reliable rapidly switching to isolate the RF generator from the catheter—means that (unlike in Embodiment 1) the catheter tip electrode E1 can be employed as an impedance measuring electrode. This is preferable, as E1 is the closest catheter electrode to the ablation zone.

For ease of depiction, only three terminals of the N-way switches are shown. The throws of switches 30 to 30X are connected to external dot electrodes 1 to N of electrode band 4. In the present embodiment, the N-way steering switches of measuring circuit 17A allow the four-terminal impedance measurements to be made in parallel, thus reducing the length of time required to measure all of the impedance paths (in this embodiment, 416 paths in total).

During the measurement phase, the I− terminal of the current source connects sequentially to each of the external dot electrodes, while the V− terminal(s) connect to the neighboring dot electrodes of the current source I− terminal location. For example, measurement circuit 17A may comprise 8 ADCs, ADC1-ADC8, with 8 corresponding N-way switches 30A to 30H, and therefore a total of 9 N-way switches (including switch 30) in switch matrix 16A. As will be understood, in this way, for each I− terminal position of AC current source 15, all 8 neighboring electrodes can be simultaneously measured, thus significantly shortening overall sampling time.

Ablation shunt relays 19A are SPDT (single-pole double-throw) relays, as used in Embodiment 1, which again operate simultaneously to either direct electrical ablation power from RF generator 12A across catheter electrode E1 and return electrode 2, or across a dummy load 25A (for example a 10 Ω resistor) while measurements are being performed. This arrangement provides protection of measuring circuit 17A and other componentry from high voltage and from RF noise.

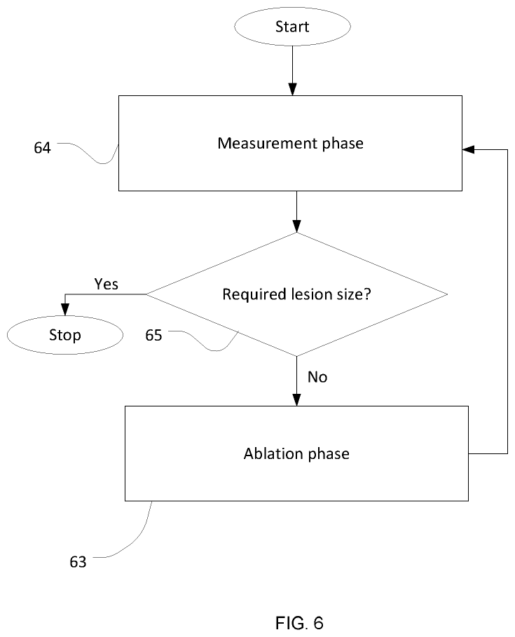

Further, ground relays 31 are arranged to operate synchronously with ablation shunt relays 19A. The ablation shunt relays 19A and ground relays 31 therefore enable the system to switch between two states, namely an ablation state and a measuring state. Once again, the method involves an iterative process of cycling between these two states, as discussed below with reference to FIG. 6.

In order to avoid noise from RF generator 12A in the impedance measurement, the relays of relay group 32 disconnect catheter electrodes E2 to E4 from the RF generator during the measurement phase. During the ablation phase however, signals from E2, E3 and E4 may be used by a physician to confirm catheter position, although position determination does not form part of the present invention.

The process of selecting 10 measurement paths described above with reference to Embodiment 1 aims to reduce measurement cycle time. This is particularly pertinent if impedance measurements may be affected by power line interference, and measurement duration must be selected to take account of such interference. For example, a duration of 5 power line cycles may be appropriate to reduce the effects of interference. With 50 Hz mains frequency, the time to take one measurement (the measurement period) may therefore be 100 ms (5×1/50 Hz). In situations where power line interference is not significant, the inventors have determined that measurement durations of 2.5 ms are suitable.

Accordingly, in the present embodiment, shorter measurement intervals coupled with the use of parallel switching allows all N electrodes to be used in each measurement phase without undesirable disruption of the ablation procedure. The specific connection pattern by which this occurs may be arranged by a sequencer so as to allow for the minimum number of changes per switch position. To this end, very rapid solid state switches are used for the N-way switches 30A-30X.

In the present embodiment, as all path impedances are measured in the measurement phase (rather than a pre-selected subset of paths), there is no need to perform a separate setup phase. The process is illustrated in FIG. 6, which depicts cycling between measurement phase 64 and ablation phase 63, determination step 65 being used to decide when to stop the ablation procedure.

### Measurement Phase

In measurement phase 64, voltage measurements resulting from the applied current are obtained and recorded for electrical paths between the selected catheter electrodes (in this case E1 and E2) and all external electrodes 1.

As described above, multiple four-terminal voltage measurements are taken for each of the external dot electrodes 1 with reference to all the neighboring electrodes. In the configuration of FIG. 8 (band 4 consisting of 64 electrodes arranged in four rows of 16), a total of 416 impedance measurements and paths are recorded (five for each electrode in the top and bottom band, and eight for each electrode in the middle bands). Where eight ADCs are used, all eight electrodes neighboring an external dot electrode 1 may be measured in a single cycle. Hence for a single measurement duration of 2.5 ms, the duration of a full measurement cycle will be 160 ms (2.5 ms×64=160 ms). As will be understood, in this scenario, three of the eight ADCs will record null measurements for top and bottom row electrodes, these null measurements automatically excluded from recordal/analysis.

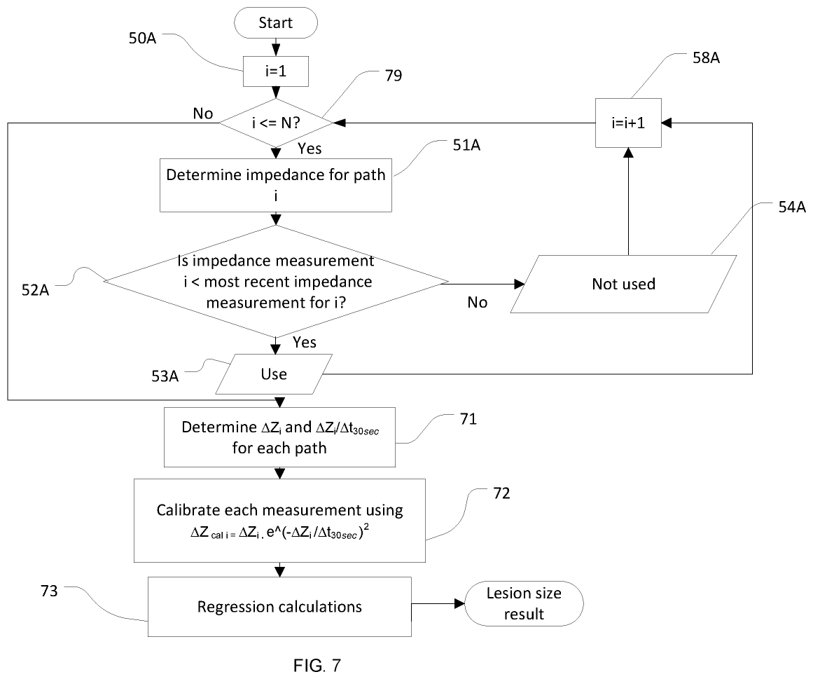

The flow diagram of FIG. 7 provides further detail of the measurement phase 64. As will be understood a number of the steps of this process are the same as described above with reference to FIG. 6 (and will not be described in detail here), however in Embodiment 2 all impedance paths are measured and processed, rather than a preselected subset of paths.

It is noted that while only results for current paths are used where the measurements show an impedance reduction (step 54A), impedance path measurement increases may also be processed in order to provide additional information. For example, such a result may signify that the catheter has moved between successive measurements.

At step 71, the difference between the previous Z_{i start} and the present Z_{i current} of each measurement is computed, ΔZᵢ. The average slope ΔZ/Δt_{30sec} (measured in Ohm/second) is measured for the first 30 seconds of ablation. At step 72 each measurement is calibrated using:

ΔZ_{cal i}=ΔZᵢ·e^{(-ΔZi/Δt}^{_{30sec}}⁾^{²}

Further suitable processing (in particular, regression analysis) is then conducted on the results to make a determination of lesion size (and other characteristics). In particular, some or all of the calibrated measurements are then used at step 73 to determine lesion size and orientation. The measurements from each cycle are regressed against time to provide a logarithmic thermal rise curve for use in determining lesion size. Orientation may be determined by correlating lesion size with external dot electrode positions. This approach removes the need for the known impedance-to-depth and impedance-to-width curves as described above with reference to Embodiment 1.

As will be understood, out of the all the impedance measurements taken, the selection of the particular impedance measurements to process (as well as the particular calibrated measurements to analyze) will depend on a variety of different factors. This selection can be made (on a dynamic basis if desired) in accordance with prescribed criteria under control of the computer software.

As schematically illustrated in FIG. 6, once the determination of lesion size following the latest ablation cycle has been made, at decision step 65 the ablation treatment process ends (if the lesion is determined to be of the required size), or (if not) the next iteration of ablation and measurement is commenced. As will be appreciated, determination step 65 may be bypassed after the initial measurement phase, before any ablation treatment has been applied.

Once ablation is discontinued, further cycles of the impedance measurement phase may be conducted, in order to monitor rebound of impedance values. While impedance change due to changes in tissue composition and architecture are permanent, those due to temperature are not. This post-ablation monitoring therefore allows generation and analysis of an impedance restitution curve, providing valuable information concerning the mechanism and characteristics of the ablation performed.

The above exemplifications of the present invention involve a four-terminal impedance measurement technique, which is a convenient approach in low impedance sensing and avoids measurement error due to contact and/or wire resistance. However the skilled reader will understand that the invention may be implemented using other techniques, such as 2- or 3-terminal approaches.

Further, in either of the embodiments described above, the impedance measurements may be gated to the respiration cycle and the ECG of the patient, to provide that measurements are taken at a relatively stable point. In this regard, a stable point is considered to be a point in time 200 ms after the first QRS following a ‘lungs empty’ indication from ventilator 100. In particular, after an ablation phase, at the next stable point generator 12 is disconnected from the catheter and switched to dummy load 25, whereupon the impedance measurements are made. The duration of a typical ablation phase may be 3-5 s (within one respiration cycle). With a patient's respiratory rate of around 12/m (i.e. 5 s), and a typical ablation procedure taking between 30 and 90 seconds, this would involve around 6-18 ablation/measurement cycles. As the skilled reader will understand, alternative approaches are possible. For example, multiple measurements can be made (for all impedance paths) for each respiration cycle, ideally gated to the patient's ECG.

As noted elsewhere in this specification, while the embodiments described and illustrated conveniently uses electrodes applied external of the patient's body to provide current conduction terminals, other sites internal of the patient's body may be suitable, so long as they are sufficiently remote from the catheter electrodes and in contact with the patient. For example, these ‘external’ electrodes may be positioned within the oesophagus and/or the coronary sinus as appropriate. In the use of such approaches, electroanatomical mapping systems may be integrated with CT/MRI imaging to accurately determine the position of these ‘external electrode’ sites within the anatomical volume.

Further, the above description involves RF ablation, but the approach can also be employed with other catheter ablation techniques, such as microwave radiation ablation. In such an embodiment, a microwave radiation catheter can be equipped with one or more suitably-positioned electrodes, such as saline electrodes or conventional metal electrodes.

As used herein, except where the context requires otherwise, the term “comprise” and variations of the term, such as “comprising”, “comprises” and “comprised”, are not intended to exclude further additives, components, integers or steps.

What is claimed is:

1. A system for monitoring tissue lesion development during a medical ablation process applied to a patient, the system comprising:
a catheter ablation device having at least one catheter electrode;
the device connectable via an electrical feedline to a source of electrical energy and configured to apply ablation energy to ablate tissue in a target region;
a plurality of external electrodes for application to the body of the patient;
measurement circuitry for determining an electrical characteristic of a current path between the at least one catheter electrode and the external electrodes in the absence of said application of ablation energy; and
an electrical controller.

2. The system of claim 1, wherein the electrical characteristic is the impedance of the current path.

3. The system of claim 2, wherein:
the electrical controller is arranged to control application of an AC current source between different combinations of the at least one catheter electrode and the plurality of external electrodes such that measurement of the resulting voltages provides a measure of impedance of different electrical paths through the body of the patient between the respective electrodes; and
wherein the electrical controller is further configured to disconnect the catheter ablation device from the source of electrical energy or otherwise suspend said application of ablation energy during application of said AC current source.

4. The system claim 1, further including a dummy resistive load for selective connection to the source of electrical energy during periods of operation of said measurement circuitry.

5. The system of claim 3, wherein the measurement circuitry further includes a switch matrix arranged for switching between the different combinations of electrodes under control of the electrical controller.

6. The system claim 1, wherein the measurement circuitry is further configured to conduct four-terminal sensing to measure the electrical characteristic.

7. The system claim 1, further including multiple analogue-to-digital converters (ADC) for simultaneous measurement of different current paths.

8. The system claim 1, wherein the source of electrical energy is an RF generator.

9. The system of claim 1, wherein the plurality of external electrodes is provided as an electrode dot harness for application across an external area of the patient's body.

10. A method of operating a system for monitoring the size of a lesion during a catheter ablation process applied to the tissue of a subject, the method comprising;
(a) performing an ablation phase involving delivery of ablation energy to a catheter electrode;
(b) performing a measure phase involving measuring an electrical characteristic of a current path passing through a lesion area formed by the ablation:
wherein ablation phase (a) and measure phase (b) are sequentially repeated.

11. The method of claim 10, wherein the ablation phase (a) and the measure phase (b) are sequentially repeated until the measurements performed in measure phase (b) indicate a prescribed lesion size.

12. The method of claim 10, wherein in the measure phase (b) ablation energy is diverted from the catheter electrode to a dummy load.

13. The method of claim 10, further including use of the system of claim 1, wherein the ablation phase (a) is conducted using said catheter ablation device, the measure phase (b) is conducted using said plurality of external electrodes and said measurement circuitry, and the switching between the ablation phase (a) and the measure phase (b) is made under control of said electrical controller.

14. The method of claim 10, further including performing an initial determination phase in which one or more current paths are selected from a plurality of current paths by applying an electrical current sequentially between one or more catheter electrodes and a plurality of electrodes applied externally of the body of a patient and measuring the electrical response, and selecting the electrodes to use for the measure phase (b) in accordance with the results.

15. The method of claim 14, wherein a prescribed number of current paths is selected in the determination phase, with the associated electrodes used for subsequent iterations of the measure phase (b).

16. The method of claim 14, wherein the electrodes are selected as those associated with the lowest impedance of the current paths measured.

17. The method of claim 14, wherein the electrodes are selected as those associated with the current paths most sensitive to a local state change of the body of the patient, such as the injection of conducting solution to a region adjacent the lesion.

18. The method according to claim 10, wherein the measurements made in measure phase (b) are used in an algorithm to estimate the size of the lesion formed in the ablation phase (a).

19. The method of claim 18 wherein, for each measure phase (b), the measurement results are analyzed and a selection is made as to which measurements to use in the algorithm.

20. The method of claim 19, wherein selection is made based at least in part on the change in the electrical characteristic of the relevant current paths since the previous measure phase (b).

21. The method of claim 10, wherein the ablation phase (a) and/or the measure phase (b) is/are gated to the respiration cycle and/or the heartbeat of the subject.
